(19) United States
(12) Patent Application Publication
(10) Pub. No.: US 2024/0358722 A1
(43) Pub. Date: Oct. 31, 2024

(54) METHODS OF ADMINISTERING FEDRATINIB

(71) Applicant: Impact Biomedicines, Inc., Summit, NJ (US)

(72) Inventors: Gopal KRISHNA, Scotch Plains, NJ (US); Michael THOMAS, Summit, NJ (US)

(21) Appl. No.: 18/692,057

(22) PCT Filed: Sep. 13, 2022
(86) PCT No.: PCT/US2022/076340

§ 371 Date: Mar. 14, 2024

Related U.S. Application Data
(60) Provisional application No. 63/331,967, filed on Apr. 18, 2022.
(60) Provisional application No. 63/243,911, filed on Sep. 14, 2021.

(51) Int. Cl. A61K 31/635; A61K 9/00; A61K 9/48; A61K 31/4178; A61K 31/51

(52) U.S. Cl. CPC A61K 31/635; A61K 9/0053; A61K 9/4825; A61K 9/4858; A61K 9/4866; A61K 31/4178; A61K 31/51

(57) ABSTRACT

The present disclosure provides methods of administering fedratinib that are useful for patients unable to take pills. In particular the present disclosure provides methods of administering fedratinib with a nutritional supplement.

### FIELD

The present disclosure relates to methods of administering fedratinib that are useful for patients unable or unwilling to take pills or capsules. The methods may also reduce or eliminate gastrointestinal discomfort associated with taking fedratinib. In particular, the present disclosure provides methods of administering fedratinib with a nutritional supplement.

### BACKGROUND

Myelofibrosis (“MF”) is a rare disease mainly affecting people of older age. MF is a BCR-ABL1-negative myeloproliferative neoplasm (“MPN”) that presents de novo (primary) or may be preceded by polycythemia vera (“PV”) or essential thrombocythemia (“ET”). Clinical features include progressive anemia, marked splenomegaly, constitutional symptoms (e.g., fatigue, night sweats, bone pain, pruritus, and cough), and weight loss. Median survival ranges from less than 2 years to over 15 years based on currently identified prognostic factors.

Fedratinib is an oral, potent small molecule inhibitor of wild type and mutationally activated Janus kinase 2 (JAK2) and FMS-like tyrosine (FLT) kinase 3 (FLT3) that is indicated for the treatment of adult patients with intermediate-2 or high-risk primary or secondary (post-polycythemia vera [PV] or post-essential thrombocythemia) myelofibrosis (MF). Fedratinib inhibits JAK2 wild-type (WT), activated mutant JAK2V617F, and FLT3 kinase, and is selective for JAK2 over JAK1, JAK3 and tyrosine kinase 2 (TYK2).

Fedratinib inhibits dysregulated JAK2 signaling that drives the pathogenesis of myeloproliferative neoplasms (MPNs), including MF and PV. In patient-derived cell lines expressing JAK2V617F and cells engineered to express WT JAK2 or JAK2V617F, fedratinib reduced phosphorylation of STAT3/STAT5, inhibited cell proliferation, and increased apoptosis. In mouse models of JAK2V617F-driven MPNs, fedratinib blocked phosphorylation of STAT⅗, increased survival, and improved disease-associated symptoms, including leukopenia, anemia, splenomegaly, and fibrosis.

As some patients have difficulty taking all four fedratinib capsules, are unable to take any medications by mouth, administering the compound in food could benefit these patients. However, some of these patients are unable to take medications with a normal diet due to the severity of their disease. Alternative methods for administering fedratinib that benefits these patients are therefore needed,

### BRIEF SUMMARY

In certain aspects, the present disclosure provides method of treating a myeloproliferative disorder in a patient in need thereof, the method comprising administering to the patient an amount effective to treat a myeloproliferative disorder, of a compound of formula (I):

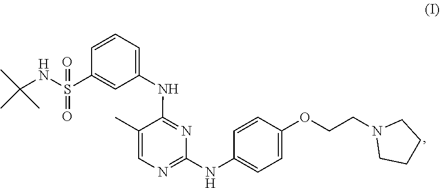 [SMILES: Cc1cnc(Nc2ccc(OCCN3CCCC3)cc2)nc1Nc1cccc(S(=O)(=O)NC(C)(C)C)c1]

or a pharmaceutical acceptable salt and/or solvate thereof and a nutritional supplement. In some aspects, the solvate is a hydrate. In some aspects, the pharmaceutically acceptable salt is a hydrochloride salt or a hydrate thereof. In some aspects, the dihydrochloride monohydrate of the compound of formula (I) is administered.

In certain aspects, the compound of formula (I), or the pharmaceutically acceptable salt and/or solvate thereof, is formulated as a capsule. In some aspects, the compound of formula (I), or the pharmaceutically acceptable salt and/or solvate thereof, is formulated by removing the contents of a capsule comprising the compound of formula (I) and dispersing it in a nutritional supplement. In some aspects, the nutritional supplement is formulated as a powder, a suspension, a paste, a gel, a pudding, a solid, a liquid, or a liquid concentrate.

In certain aspects, the compound of formula (I), or the pharmaceutically acceptable salt and/or solvate thereof, and the nutritional supplement are administered separately. In some aspects, the compound of formula (I), or the pharmaceutically acceptable salt and/or solvate thereof, and the nutritional supplement are administered sequentially within a time period of 15 minutes or less.

In certain aspects, the compound of formula (I), or the pharmaceutically acceptable salt and/or solvate thereof, and the nutritional supplement are administered concomitantly.

In some aspects, the compound of formula (I), or the pharmaceutically acceptable salt and/or solvate thereof, is dispersed in the nutritional supplement.

In some aspects, the nutritional supplement is formulated as a liquid.

In some aspects, the compound of formula (I), or the pharmaceutically acceptable salt and/or solvate thereof, is formulated as a powder.

In certain aspects, the compound of formula (I), or the pharmaceutically acceptable salt and/or solvate thereof, is administered through a nasogastric tube. In some aspects, the compound of formula (I), or the pharmaceutically acceptable salt and/or solvate thereof, and the nutritional supplement are administered through a nasogastric tube.

In certain aspects, the compound of formula (I), or the pharmaceutically acceptable salt and/or solvate thereof, and the nutritional supplement are administered orally.

In some aspects, the method further comprises administering an anti-emetic compound. In some aspects, the anti-emetic compound is selected from droperidol, granisetron, metoclopramide, dexamethasone, bismuth subsalicylate, ondansetron, aprepitant, dolasetron, palonosetron, prochlorperazine, and rolapitant. In some aspects, the anti-emetic compound is ondansetron. In some aspects, the anti-emetic compound is administered about 1 hour before the administration of the compound of formula (I), or the pharmaceutically acceptable salt and/or solvate thereof, and the nutritional supplement. In some aspects, the anti-emetic compound is administered at a dose of about 8 mg. In some aspects, at least one additional dose of the anti-emetic compound is administered after the administration of the compound of formula (I), or the pharmaceutically acceptable salt and/or solvate thereof and the nutritional supplement.

In certain aspects, the compound of formula (I), or the pharmaceutically acceptable salt and/or solvate thereof, is administered at a dose from about 50 mg to about 500 mg. In some aspects, the compound of formula (I), or the pharmaceutically acceptable salt and/or solvate thereof, is administered at a dose of about 400 mg per day. In some aspects, the compound of formula (I), or the pharmaceutically acceptable salt and/or solvate thereof, is administered once daily. In some aspects, the compound of formula (I), or the pharmaceutically acceptable salt and/or solvate thereof, is administered twice daily. In some aspect, the compound of formula (I), or the pharmaceutically acceptable salt and/or solvate thereof, is administered at a dose of about 200 mg twice daily.

In some aspects, the nutritional supplement does not comprise thiamine. In some aspects, the nutritional supplement comprises thiamine. In some aspects, the nutritional supplement contains one or more of vitamins, minerals, proteins, fats, and carbohydrates.

In some aspects, the nutritional supplement is a beverage. In some aspects, the beverage is a shake.

In some aspects, the patient is administered a compound of formula (Ia):

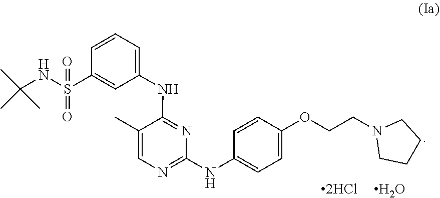

In certain aspects, the myeloproliferative disorder is myelofibrosis. In some aspects, the myelofibrosis is primary myelofibrosis. In certain aspects, the primary myelofibrosis is selected from intermediate risk primary myelofibrosis and high-risk primary myelofibrosis. In some aspects, the myelofibrosis is secondary myelofibrosis. In some aspects, the myelofibrosis is post-essential thrombocythemia myelofibrosis. In some aspects, the myelofibrosis is post-polycythemia vera myelofibrosis.

In some aspects, the myeloproliferative disorder is acute myeloid leukemia

(AML).

In certain aspects, the myeloproliferative disorder is polycythemia vera.

In some aspects, the myeloproliferative disorder is essential thrombocythemia.

### BRIEF DESCRIPTION OF THE FIGURES

FIG. 1 shows the study design for Part 1 of a clinical trial. ET means early termination; Trt means treatment. Trt 1A refers to treatment with fedratinib 4×100 mg capsules plus nutritional supplement; Trt 1B refers to treatment with fedratinib 400 mg dispersed in nutritional supplement.

FIG. 2 shows the study design for Part 2 of a clinical trial. ET means early termination; Trt means treatment. Trt 2A refers to treatment with fedratinib 4×100 mg capsules plus nutritional supplement; Trt 2B refers to treatment with fedratinib 400 mg in nutritional supplement administered via NG tube; Trt 2C refers to treatment with fedratinib 2×100 mg capsules BID with nutritional supplement.

### DETAILED DESCRIPTION

Provided are formulations and compositions comprising a therapeutically effective amount of compound of formula (I)

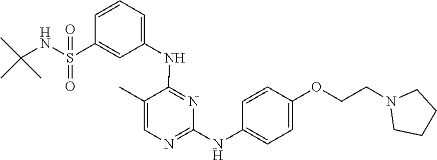 [SMILES: Cc1cnc(Nc2ccc(OCCN3CCCC3)cc2)nc1Nc1cccc(S(=O)(=O)NC(C)(C)C)c1]

or a pharmaceutical acceptable salt and/or solvate thereof, and a nutritional supplement.

### Definitions

In order that the present description can be more readily understood, certain terms are first defined. Additional definitions are set forth throughout the detailed description.

It is to be noted that the term “a” or “an” entity refers to one or more of that entity; for example, “a nutritional supplement,” is understood to represent one or more nutritional supplements. As such, the terms “a” (or “an”), “one or more,” and “at least one” can be used interchangeably herein.

As used herein, unless specifically indicated otherwise, the word “or” is used in the inclusive sense of “and/or” and not the exclusive sense of “either/or.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some aspects of the disclosure, the term “about” encompasses a deviation from the recited value of between 0.001% and 10%, inclusive of the endpoints. In some aspects, the term “about” encompasses an increase from the recited value of between 0.001% and 10%, inclusive of the endpoints. In some aspects, the term “about” encompasses a decrease from the recited value of between 0.001% and 10%, inclusive of the endpoints.

Units, prefixes, and symbols are denoted in their Systéme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each sub-combination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

As used herein, the terms “effective amount” and “therapeutically effective amount” refer to an amount of an agent (e.g., fedratinib) that provides beneficial or desired therapeutic and/or prophylactic results. For prophylactic use, beneficial or desired results can include, for example, one or more results such as eliminating or reducing the risk, lessening the severity, or delaying the onset of the disease, including biochemical, histological, and/or behavioral symptoms of the disease, its complications, and intermediate pathological phenotypes presenting during development of the disease. For therapeutic use, beneficial or desired results can include, for example, one or more clinical results such as decreasing one or more symptoms and pathological conditions resulting from or associated with the disease, increasing the quality of life of those suffering from the disease, decreasing the dose of other medications required to treat the disease, enhancing the effect of other medication such as via targeting, delaying the progression of the disease, and/or prolonging survival. An effective amount can be, for example, an amount sufficient to accomplish prophylactic or therapeutic treatment either directly or indirectly. As it is understood in the clinical context, an effective amount of a drug, compound, or pharmaceutical composition may or may not be achieved in conjunction with another drug, compound, or pharmaceutical composition. Thus, an effective amount may be considered in the context of administering one or more therapeutic agents. An effective amount can be administered in one dosage or can be divided into multiple dosages, the total of such dosages being the effective amount. For example, an effective amount can be provided in two separate administrations over a period of time, that in aggregate, provide the effective amount of the formulation.

In certain aspects, the effective amount of fedratinib is the amount clinically proven to treat a myeloproliferative disorder such as myelofibrosis. The effective amount of fedratinib can have the effect in reducing one or more of splenomegaly, improving constitutional symptoms (such as early satiety, fatigue, night sweats, cough, and pruritus), reducing leukocytosis, reducing thrombocytosis, decreasing JAK2V617F allele burden, reducing bone marrow fibrosis, and/or reducing bone marrow cellularity.

The term “subject” refers to a human. The terms “subject” and “patient” are used interchangeably herein.

The term “concomitant” refers to the administration of the compound of formula (I), or a pharmaceutically acceptable salt and/or solvate thereof, and the nutritional supplement at the same time or almost at the same time. For example, the compound of formula (I), or a pharmaceutically acceptable salt and/or solvate thereof, and the nutritional supplement are administered “almost at the same time,” when they are administered no more than about 15, 14, 13, 12, 11, 10, 9, 8, 7, 6, 5, 4, 3, 2 minutes, or 1 minute apart.

The terms “sequential” and “sequentially” refer to the administration of the compound of formula (I), or a pharmaceutically acceptable salt and/or solvate thereof, and a nutritional supplement, as disclosed herein, to a patient at two different time points that are separated by 16 minutes or more, for example, 18 minutes, 20 minutes, 25 minutes, 30 minutes, 35 minutes, 40 minutes, 45 minutes, 50 minutes, 55 minutes, 60 minutes, 70 minutes, 80 minutes or 90 minutes or more.

### Methods of Treatment

In certain aspects, the present disclosure provides methods of treating a myeloproliferative disorder in a patient in need thereof by administering a compound of formula (I), or a pharmaceutically acceptable salt and/or solvate thereof, and a nutritional supplement.

In certain aspects, the compound of formula (I), or the pharmaceutically acceptable salt and/or solvate thereof, can be administered orally. In some aspects, the compound of formula (I), or the pharmaceutically acceptable salt and/or solvate thereof, can be administered as a capsule. In some aspects, the compound of formula (I), or the pharmaceutically acceptable salt and/or solvate thereof, can be administered as a tablet. In some aspects, the compound of formula (I), or the pharmaceutically acceptable salt and/or solvate thereof, can be administered as a powder.

In certain aspects, the compound of formula (I), or the pharmaceutically acceptable salt and/or solvate thereof, can be administered separately. For example, the compound of formula (I), or a pharmaceutically acceptable salt and/or solvate thereof, and the nutritional supplement can be administered at two different time points that are separated by 16 minutes, 18 minutes, 20 minutes, 25 minutes, 30 minutes, 35 minutes, 40 minutes, 45 minutes, and/or 50 minutes.

In some aspects, the nutritional supplement can be administered before the compound of formula (I), or a pharmaceutically acceptable salt and/or solvate thereof. In some aspects, the nutritional supplement can be administered within a time period of 15 minutes or less before the compound of formula (I), or a pharmaceutically acceptable salt and/or solvate thereof. In some aspects, the nutritional supplement can be administered 16 minutes, 18 minutes, 20 minutes, 25 minutes, 30 minutes, 35 minutes, 40 minutes, 45 minutes, and/or 50 minutes before the compound of formula (I), or a pharmaceutically acceptable salt and/or solvate thereof.

In some aspects, the compound of formula (I), or pharmaceutically acceptable salt and/or solvate thereof and the nutritional supplement can be administered concomitantly. In some aspects, the compound of formula (I), or pharmaceutically acceptable salt and/or solvate thereof and the nutritional supplement can be administered within a time period of 15 minutes or less.

In some aspects, the compound of formula (I), or the pharmaceutically acceptable salt and/or solvate thereof, can be dispersed in the nutritional supplement. In certain aspects, the compound of formula (I), or the pharmaceutically acceptable salt and/or solvate thereof, can be dispersed in the form of a powder. In certain aspects, the compound of formula (I), or the pharmaceutically acceptable salt and/or solvate thereof, can be dispersed as the contents of a capsule. In some aspects, the nutritional supplement can be formulated as a powder, a suspension, a paste, a gel, a pudding, a solid, a liquid, or a liquid concentrate. In some aspects, the nutritional supplement can be formulated as a liquid.

In some aspects, the nutritional supplement can be formulated as a beverage. In some aspects, the beverage can be selected from the group consisting of a carbonated beverage, a shake, a powdered concentrate, a liquid concentrate, a fruit juice, a fruit juice-flavored drink, a fruit-flavored drink, a sports drink, an energy drink, a fortified/enhanced water drink, a soy drink, a vegetable drink, a grain-based drink, a malt beverage, a fermented drink, a yogurt drink, kefir, a coffee beverage, a tea beverage, a dairy beverage, and mixtures of any of them. In some aspects, the nutritional supplement can be formulated as a shake.

In some aspects, the compound of formula (I), or the pharmaceutically acceptable salt and/or solvate thereof, the mixture of the compound of formula (I), or the pharmaceutically acceptable salt and/or solvate thereof, and the nutritional supplement can be administered to the subject orally. In some aspects, the compound of formula (I), or the pharmaceutically acceptable salt and/or solvate thereof, the mixture of the compound of formula (I), or the pharmaceutically acceptable salt and/or solvate thereof, and the nutritional supplement can be administered to the subject through a nasogastric tube.

In certain aspects, the compound of formula (I), or a pharmaceutically acceptable salt and/or solvate thereof and the nutritional supplement are administered with an anti-emetic compound. The term “anti-emetic compound” refers to compounds that treat or alleviate nausea and/or vomiting and include but are not limited to, droperidol, granisetron, metoclopramide, dexamethasone, bismuth subsalicylate, ondansetron, aprepitant, dolasetron, palonosetron, prochlorperazine, or rolapitant. In some aspects, the anti-emetic compound can be ondansetron.

In some aspects, the anti-emetic compound can be administered before the compound of formula (I), or a pharmaceutically acceptable salt and/or solvate thereof and the nutritional supplement. For example, the anti-emetic compound can be administered between about 1 minute and about 80 minutes before the compound of formula (I), or a pharmaceutically acceptable salt and/or solvate thereof and the nutritional supplement. In some aspects, the anti-emetic compound can be administered about 1 hour before the compound of formula (I), or a pharmaceutically acceptable salt and/or solvate thereof and the nutritional supplement.

In some aspects, the anti-emetic compound can be administered concomitantly with the compound of formula (I), or a pharmaceutically acceptable salt and/or solvate thereof and the nutritional supplement.

In some aspects, the anti-emetic compound can be administered at a single dose of 8 mg. In some aspects, the anti-emetic compound can be administered at a dose of about 8 mg about 1 hour before the compound of formula (I), or a pharmaceutically acceptable salt and/or solvate thereof and the nutritional supplement.

In some aspects, at least one additional dose of the anti-emetic compound can be administered after the administration of the compound of formula (I), or the pharmaceutically acceptable salt and/or solvate thereof and the nutritional supplement. In some aspects, at least one additional dose of 8 mg of ondansetron can be administered after the administration of the compound of formula (I), or the pharmaceutically acceptable salt and/or solvate thereof and the nutritional supplement.

In some aspects, the compound of formula (I), or the pharmaceutically acceptable salt and/or solvate thereof, can be administered at an effective amount for the method. In some embodiments, the compound of formula (I), or the pharmaceutically acceptable salt and/or solvate thereof, can be administered at a dose less than a standard dose. In some aspects, a standard dose of the compound of formula (I), or the pharmaceutically acceptable salt and/or solvate thereof, is about 400 mg/day. In some embodiments, the compound of formula (I), or the pharmaceutically acceptable salt and/or solvate thereof, can be administered at a dose that is about ⅛ a standard dose. In some embodiments, the compound of formula (I), or the pharmaceutically acceptable salt and/or solvate thereof, can be administered at a dose that is about ¼ a standard dose. In some embodiments, the compound of formula (I), or the pharmaceutically acceptable salt and/or solvate thereof, can be administered at a dose that is about ½ a standard dose.

In some aspects, the compound of formula (I), or the pharmaceutically acceptable salt and/or solvate thereof can be administered at from about 50 mg to about 700 mg, from about 75 mg to about 300 mg, or from about 90 mg to about 200 mg. In some aspects, the compound of formula (I), or the pharmaceutically acceptable salt and/or solvate thereof, can be administered at about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 550 mg, about 600 mg, about 650 mg, or about 700 mg, or an amount ranging from and to any of these values. In some aspects, the compound of formula (I), or the pharmaceutically acceptable salt and/or solvate thereof, can be administered at about 50 mg. In some aspects, the compound of formula (I), or the pharmaceutically acceptable salt and/or solvate thereof, can be administered at about 100 mg. In some aspects, the compound of formula (I), or the pharmaceutically acceptable salt and/or solvate thereof, can be administered at about 200 mg. In some aspects, the compound of formula (I), or the pharmaceutically acceptable salt and/or solvate thereof, can be administered at about 300 mg. In some aspects, the compound of formula (I), or the pharmaceutically acceptable salt and/or solvate thereof, can be administered at about 400 mg. Fedratinib is available in the U.S. as INREBIC®, and is available as capsules having 100-mg of fedratinib in hard gelatin capsules. The capsule contents are manufactured with 117.3 mg of fedratinib dihydrochloride monohydrate.

In some aspects, the compound of formula (I), or the pharmaceutically acceptable salt and/or solvate thereof, can be administered from 1 to 10 times a day. In some aspects, the compound of formula (I), or the pharmaceutically acceptable salt and/or solvate thereof, can be administered 1 to 5 times a day. In some aspects, the compound of formula (I), or the pharmaceutically acceptable salt and/or solvate thereof, can be administered 1, 2, 3, 4, 5, 6, 7, 8, 9, or 10 times a day. In some aspects, the compound of formula (I), or the pharmaceutically acceptable salt and/or solvate thereof, can be administered once a day. In some aspects, the compound of formula (I), or the pharmaceutically acceptable salt and/or solvate thereof, can be administered every other day. In some aspects, the compound of formula (I), or the pharmaceutically acceptable salt and/or solvate thereof, can be administered once every other day. In some aspects, the compound of formula (I), or the pharmaceutically acceptable salt and/or solvate thereof, can be administered every two days. In some aspects, the compound of formula (I), or the pharmaceutically acceptable salt and/or solvate thereof, can be administered once every two days.

In some aspects, the compound of formula (I), or the pharmaceutically acceptable salt and/or solvate thereof, can be administered at 50 mg once a day. In some aspects, the compound of formula (I), or the pharmaceutically acceptable salt and/or solvate thereof, can be administered at 100 mg once a day. In some aspects, the compound of formula (I), or the pharmaceutically acceptable salt and/or solvate thereof, can be administered at 100 mg once every other day. In some aspects, the compound of formula (I), or the pharmaceutically acceptable salt and/or solvate thereof, can be administered at 200 mg once a day. In some aspects, the compound of formula (I), or the pharmaceutically acceptable salt and/or solvate thereof, can be administered at 200 mg twice a day. In some aspects, the compound of formula (I), or the pharmaceutically acceptable salt and/or solvate thereof, can be administered at 200 mg once every other a day. In some aspects, the compound of formula (I), or the pharmaceutically acceptable salt and/or solvate thereof, can be administered at 300 mg once a day. In some aspects, the compound of formula (I), or the pharmaceutically acceptable salt and/or solvate thereof, can be administered at 300 mg once every other day. In some aspects, the compound of formula (I), or the pharmaceutically acceptable salt and/or solvate thereof, can be administered at 400 mg once a day. In some aspects, the compound of formula (I), or the pharmaceutically acceptable salt and/or solvate thereof, can be administered at 400 mg once every other day.

In some aspects, the compound of formula (Ia) can be administered at 50 mg once a day. In some aspects, the compound of formula (Ia) can be administered at 100 mg once a day. In some aspects, the compound of formula (Ia) can be administered at 100 mg once every other day. In some aspects, the compound of formula (Ia) can be administered at 200 mg once a day. In some aspects, the compound of formula (I), or the pharmaceutically acceptable salt and/or solvate thereof, can be administered at 200 mg twice a day. In some aspects, the compound of formula (Ia) can be administered at 200 mg once every other a day. In some aspects, the compound of formula (Ia) can be administered at 300 mg once a day. In some aspects, the compound of formula (Ia) can be administered at 300 mg once every other day. In some aspects, the compound of formula (I), or the pharmaceutically acceptable salt and/or solvate thereof, can be administered at 300 mg once every other day. In some aspects, the compound of formula (I), or the pharmaceutically acceptable salt and/or solvate thereof, can be administered at 400 mg once a day. In some aspects, the compound of formula (I), or the pharmaceutically acceptable salt and/or solvate thereof, can be administered at 400 mg once every other day.

In some aspects, the myeloproliferative disorder treated by the compound of formula (I), or the pharmaceutically acceptable salt and/or solvate thereof, is myelofibrosis. In aspects, the myeloproliferative disorder can be myelofibrosis. In some aspects, the myelofibrosis can be primary myelofibrosis. In some aspects, the primary myelofibrosis can be Dynamic International Prognostic Scoring System (DIPSS) intermediate or high-risk primary myelofibrosis. In some aspects, the myelofibrosis can be secondary myelofibrosis. In some aspects, the myelofibrosis can be post-essential thrombocythemia myelofibrosis. In some aspects, the myelofibrosis can be post-polycythemia vera myelofibrosis. In some aspects, the myeloproliferative disorder can be polycythemia vera. In some aspects, the myeloproliferative disorder can be essential thrombocythemia. In some aspects, the myeloproliferative disorder can be acute myeloid leukemia (AML).

In certain aspects, the present disclosure provides methods of improving the safety and/or tolerability of a compound of formula (I), or a pharmaceutically acceptable salt and/or solvate thereof in a patient concurrently receiving a dual CYP2C19 and CYP3A4 inhibitor. In some aspects, improved safety and/or tolerability comprises a reduction in one or more conditions selected from anemia, thrombocytopenia, gastrointestinal toxicity, hepatic toxicity, amylase elevation, and lipase elevation.

In some aspects, the patient can be administered thiamine or a thiamine equivalent prior to, simultaneously with, or after the compound of formula (I), or the pharmaceutically acceptable salt and/or solvate thereof. In some aspects, the thiamine or thiamine equivalent can be administered orally. In some aspects, the thiamine or thiamine equivalent can be administered intravenously. Methods of administering thiamine or thiamine equivalents with the compound of formula (I), or the pharmaceutically acceptable salt and/or solvate thereof, are disclosed in WO 2020/068755, which is incorporated by reference in its entirety.

In certain aspects, the patient can be administered a 5-HT3 receptor antagonist prior to, simultaneously with, or after the compound of formula (I), or a pharmaceutically acceptable salt and/or solvate thereof. In some aspects, the 5-HT3 receptor antagonist can be selected from ondansetron, granisetron, dolasetron, and palonosetron. In certain aspects, the 5-HT3 receptor antagonist is ondansetron.

In some aspects, the subject is less than 18 years old. In some aspects, the subject is 18 years or older. In certain aspects, the subject is between 18 years of age and 29 years of age, inclusive of the endpoints. In some aspects, the subject is between 30 years of age and 49 years of age, inclusive of the endpoints. In some aspects, the subject is between 50 years of age and 69 years of age, inclusive of the endpoints. In certain aspects, the subject is between 70 years of age and 100 years of age, inclusive of the endpoints. In certain aspects, the subject is between 18 years of age and 69 years of age.

In some aspects, the compound of formula (I), or the pharmaceutically acceptable salt and/or solvate thereof, can be administered to the subject prior to or after the administration of the dual CYP2C19 and CYP3A4 inhibitor. In other aspects, the compound of formula (I), or the pharmaceutically acceptable salt and/or solvate thereof, can be administered to the subject concurrently with the dual CYP2C19 and CYP3A4 inhibitor.

### Compound of Formula (I):

The present disclosure provides methods of treating a myeloproliferative disorder comprising administering to a patient in need thereof an effective amount of a compound of formula (I), or a pharmaceutically acceptable salt and/or solvate thereof, wherein the patient is concurrently receiving a dual CYP2C19 and CYP3A4 inhibitor. Also provided is a method of increasing the safety and/or tolerability of a compound of formula (I), or a pharmaceutically acceptable salt and/or solvate thereof in a patient concurrently receiving a dual CYP2C19 and CYP3A4 inhibitor.

“Pharmaceutically acceptable salts” are derived from inorganic or organic acids or bases. Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts,

N-methyl-D-glucamine; and salts with amino acids such as arginine, lysine, and the like. For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2-ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′-di (dehydroabietyl)-ethylenediamine), hydrobromide, hydrochloride, hydroxynaphthoate, iodide, isethionate (2-hydroxyethanesulfonate), lactate, lactobionate, malate, maleate, mandelate, mesylate (methanesulfonate), methylbromide, methylnitrate, methylsulfate, mucate, napsylate (2-naphthalenesulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′-dibenzylethylenediamine), chloroprocaine, choline, diethanolamine, ethylenediamine, meglumine (N-methylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis (salicylate), napadisylate (1,5-naphthalenedisulfonate), oxalate, pectinate, persulfate, phenylethylbarbiturate, picrate, propionate, thiocyanate, tosylate and undecanoate; organic cations benethamine (N-benzylphenethylamine), clemizole (1-p-chlorobenzyl-2-pyrrolildine-1′-ylmethylbenzimidazole), diethylamine, piperazine and tromethamine (tris (hydroxymethyl)aminomethane); and metallic cations barium and bismuth.

A “solvate” refers to a physical association of a compound of formula (I) with one or more solvent molecules, whether organic or inorganic. This physical association includes hydrogen bonding. In certain instances, the solvate will be capable of isolation, for example when one or more solvent molecules are incorporated in the crystal lattice of the crystalline solid. Examples of solvates include, but are not limited to, hydrates, ethanolates, methanolates, isopropanolates, acetonitrile solvates, and ethyl acetate solvates. Methods of solvation are known in the art.

In some aspects, the compound of formula (I) can be present as a hydrate, which can be obtained, for example, by crystallization from a solvent or from aqueous solution. Any arbitrary number of solvate or water molecules can combine with the compound of formula (I) to form solvates and hydrates. Unless stated to the contrary, the disclosure includes all such possible solvates. Examples of solvates of the compound of formula (I) are disclosed in WO 2020/167845, which is hereby incorporated by reference.

In certain aspects, the compound of formula (I) is a compound of formula (Ia):

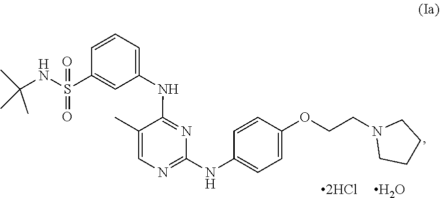

known as fedratinib, which has the chemical name N-tert-butyl-3-[(5-methyl-2-{[4-(2-pyrrolidin-1-ylethoxy)phenyl]amino} pyrimidin-4-yl)amino] benzenesulfonamide dihydrochloride monohydrate, and has been previously described, e.g., in U.S. Pat. Nos. 7,528,143; 7,825,246; 8,138,199; and 10,391,094; and in PCT Application Publication Nos. WO 2020/167845 and WO 2020/068755, which are each hereby incorporated by reference in their entireties.

### Nutritional Supplement

The present disclosure provides nutritional supplements that are administered with a JAK inhibitor, specifically a compound of formula (I), or a pharmaceutically acceptable salt and/or solvate thereof. The term “nutritional supplement” refers to a preparation intended to supplement the diet and provide nutrients, such as carbohydrates, fatty acids, amino acids, vitamins, minerals, and/or fiber to a subject. A nutritional supplement can be a substance or formulation that satisfies at least a portion of a subject's nutrient requirements and, thus may or may not be the sole source of nutrition. A nutritional supplement can be formulated in ways known to the skilled artisan, such as, but not limited to, a powder, a suspension, a paste, a gel, a pudding, a solid, a liquid, a liquid concentrate. A nutritional supplement can be formulated into a ready-to-use product. A nutritional supplement can be designed to provide adequate amounts and combination of micronutrients and macronutrient along with optimized energy content. A “nutritionally complete” nutritional supplement provides adequate amounts of all carbohydrates, lipids, essential fatty acids, proteins, essential amino acids, conditionally essential amino acids, vitamins, minerals, and energy required for normal growth and/or weight maintenance. The principal micronutrients fall into two categories: vitamins and minerals. Vitamins are essential micronutrients that the body is not capable of synthesizing in sufficient quantities for its growth and maintenance and have to be derived from dietary food sources and comprise four, A, D, E, and K fat soluble vitamins and nine, B1, B2, B3, B6, B12, pantothenic acid, biotin, folic acid, and C water soluble vitamins. Minerals include macrominerals such as calcium (Ca), phosphorous (P), sodium (Na), potassium (K), magnesium (Mg), and chloride (Cl) and trace minerals such as iron (Fe), zinc (Zn), iodine (Io), selenium (Se), copper (Cu), manganese (Mn), fluoride (F), chromium (Cr) and molybdenum (Mo). In some aspects, the nutritional supplement does not contain thiamine. In some aspects, the nutritional supplement does contain thiamine. The vitamins, minerals, proteins, fats, and carbohydrates included in the nutritional supplements can be used in any suitable form, for example, a powder, a suspension, a paste, a gel, a pudding, a solid, a liquid, or a liquid concentrate. The nutritional supplement can be a commercially-available nutritional supplement including, but not limited to, Ensure Plus® or Boost Plus®. A nutritional supplement may be provided in liquid form, or as a powder for reconstitution with a liquid (e.g., water).

In some embodiments, the nutritional supplement is not a supplement that solely contains thiamine as the active ingredient.

In certain aspects, the nutritional supplement can be administered in a single dose of about 180 mL. In certain aspects, the nutritional supplement can be administered in multiple doses. In certain aspects, the nutritional supplement can be administered in an amount sufficient to deliver the compound of formula (I), or a pharmaceutically acceptable salt or solvate thereof, with sufficient palatability.

The term “powder” refers to any form of dry material. For example, powder nutritional supplements are typically in the form of flowable or substantially flowable particulate compositions or at least particulate compositions that can be easily scooped and measured with a spoon or similar other device, wherein the compositions can easily be reconstituted with a suitable aqueous fluid, typically water, to form a liquid nutritional formula for immediate oral use. In this context, “immediate” use generally means within about 48 to 40 hours, typically within about 24 hours, most typically right after reconstitution. Nutritional supplement powders include spray dried, agglomerated, dry mixed or other known or otherwise effective particulate forms.

The term “dispersion” refers to a material that comprises one or more phases where at least one of the phases consists of finely divided phase domains, often in the colloidal size range, distributed throughout a continuous phase. A dispersion can be classified according to the size of the particles of the two phases, whether or not precipitation occurs, and according to the presence of Brownian motion. Dispersion is a process by which, in case of a solid dispersed in a liquid, agglomerated particles are separated from each other and a new interface between the inner surface of the liquid dispersion medium and the surface of the dispersed particles is generated. This process is facilitated by molecular diffusion and convection. A “solution” refers to a homogenous mixture where the dispersed particles will not settle if the solution is left undisturbed for a prolonged period of time. A “colloid” refers to a homogeneous non-crystalline substance consisting of large molecules or ultramicroscopic particles of one substance dispersed through a second substance. Colloids include gels, sols, and emulsions; the particles do not settle, and cannot be separated out by ordinary filtering or centrifuging like those in a suspension. A “suspension” is a heterogeneous dispersion of large particles in a medium. Unlike solutions and colloids, if left undisturbed for a prolonged period of time, suspended particles will settle out of the mixture.

The term “paste” refers to a thick, soft, moist substance typically produced by mixing dry ingredients with a liquid.

The term “pudding” refers to a mixture of ingredients with a grain product or other binder such as butter, flour, cereal, and/or eggs resulting in a solid mass.

### Compositions of Compound of Formula (I)

Pharmaceutical compositions comprising the compound of formula (I) can also comprise suitable carriers, excipients, and auxiliaries that may differ depending on the mode of administration.

The compound of formula (I) can be formulated for administration by a variety of means including orally, parenterally, by inhalation spray, topically, or rectally in formulations containing pharmaceutically acceptable carriers, adjuvants, and vehicles. The term “parenteral” as used here includes subcutaneous, intravenous, intramuscular, and intraarterial injections with a variety of infusion techniques. Intraarterial and intravenous injection as used herein includes administration through catheters.

The compounds of the disclosure and the compositions of the disclosure can be formulated in accordance with the routine procedures adapted for desired administration route. Accordingly, the compositions of the disclosure can take such forms as suspensions, solutions or emulsions in oily or aqueous vehicles, and can contain formulatory agents such as suspending, stabilizing and/or dispersing agents. The compounds of the disclosure and the compositions of the disclosure can be formulated as a preparation suitable for implantation or injection. Thus, for example, the compound of formula (I) can be formulated with suitable polymeric or hydrophobic materials (e.g., as an emulsion in an acceptable oil) or ion exchange resins, or as sparingly soluble derivatives (e.g., as a sparingly soluble salt). The compounds of the disclosure and the compositions of the disclosure can be in powder form for constitution with a suitable vehicle, e.g., sterile pyrogen-free water, before use. Suitable formulations for each of these methods of administration can be found, for example, in Remington: The Science and Practice of Pharmacy, A Gennaro, ed., 20th edition, Lippincott, Williams & Wilkins, Philadelphia, PA

In some aspects, the compositions of the disclosure are suitable for oral administration. These compositions can comprise solid, semisolid, gel matrix or liquid dosage forms suitable for oral administration. As used herein, oral administration includes buccal, lingual, and sublingual administration. Suitable oral dosage forms include, without limitation, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups or any combination thereof. In some aspects, compositions of the disclosure suitable for oral administration are in the form of a tablet or a capsule. In some aspects, the compound of the disclosure can be in the form of a capsule. In some aspects, capsules can be immediate release capsules.

The compositions of the disclosure can be in the form of compressed tablets, tablet triturates, chewable lozenges, rapidly dissolving tablets, multiple compressed tablets, or enteric-coated tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which can be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. A film coating can impart the same general characteristics as a sugar coating. Multiple compressed tablets are compressed tablets made by more than one compression cycle, including layered tablets, and press-coated or dry-coated tablets.

In some aspects, the compound of the disclosure can be in the form of a tablet. In some aspects, the compound of the disclosure can be in the form of a compressed tablet. In some aspects, the compound of the disclosure can be in the form of a film-coated compressed tablet. In some aspects, the compositions of the disclosure can be in the form of film-coated compressed tablets.

In some aspects, the compositions of the disclosure can be prepared by fluid bed granulation of the compound of the disclosure with one or more pharmaceutically acceptable carriers, vehicles, and/or excipients. In some aspects, the compositions of the disclosure can be prepared by fluid bed granulation process and can provide a tablet formulation with good flowability, good compressibility, fast dissolution, good stability, and/or minimal to no cracking. In some aspects, the fluid bed granulation process can allow preparation of formulations having high drug loading, such as over 70% or over 75% of a compound of the disclosure.

In some aspects, the compositions of the disclosure can be in the form of soft or hard capsules, which can be made from gelatin, methylcellulose, starch, and/or calcium alginate. The hard gelatin capsule, also known as the dry-filled capsule (DFC), can comprise two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. In some aspects, soft gelatin shells can contain a preservative to prevent the growth of microorganisms. Suitable preservatives include, but are not limited to, those as described herein, including methyl—and propyl-parabens, sorbic acid, and combinations thereof. The liquid, semisolid, and solid dosage forms provided herein can be encapsulated in a capsule. Suitable liquid and semisolid dosage forms include, but are not limited to, solutions and suspensions in propylene carbonate, vegetable oils, triglycerides, and combinations thereof. Capsules containing such solutions can be prepared as described in U.S. Pat. Nos. 4,328,245; 4,409,239; and 4,410,545. The capsules can also be coated as known by those of skill in the art in order to modify or sustain dissolution of the active ingredient.

In some aspects, the compositions of the disclosure can be in liquid or semisolid dosage forms, including emulsions, solutions, suspensions, elixirs, and syrups. In some aspects, the emulsion can be a two-phase system, in which one liquid is dispersed in the form of small globules throughout another liquid, which can be oil-in-water or water-in-oil. Emulsions can include a pharmaceutically acceptable non-aqueous liquids or solvent, emulsifying agent, and preservative. Suspensions can include a pharmaceutically acceptable suspending agent and preservative. Aqueous alcoholic solutions can include a pharmaceutically acceptable acetal, such as a di-(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs can be clear, sweetened, and hydroalcoholic solutions. Syrups can be concentrated aqueous solutions of a sugar, for example, sucrose, and can comprise a preservative. For a liquid dosage form, for example, a solution in a polyethylene glycol can be diluted with a sufficient quantity of a pharmaceutically acceptable liquid carrier, e.g., water, to be measured conveniently for administration.

In some aspects, the compositions of the disclosure for oral administration can be also provided in the forms of liposomes, micelles, microspheres, or nanosystems. Micellar dosage forms can be prepared as described in U.S. Pat. No. 6,350,458.

In some aspects, the compositions of the disclosure can be provided as non-effervescent or effervescent, granules and powders, to be reconstituted into a liquid dosage form. Pharmaceutically acceptable carriers and excipients used in the non-effervescent granules or powders can include, but are not limited to, diluents, sweeteners, wetting agents, and mixtures thereof. Pharmaceutically acceptable carriers and excipients used in the effervescent granules or powders can include, but are not limited to, organic acids, a source of carbon dioxide, and mixtures thereof.

Coloring and flavoring agents can be used in all of the above dosage forms. In addition, flavoring and sweetening agents can be especially useful in the formation of chewable tablets and lozenges.

In certain aspects, the compositions of the disclosure can be formulated as immediate or modified release dosage forms, including delayed-, extended, pulsed-, controlled, targeted-, and programmed-release forms.

The compositions of the disclosure can comprise another active ingredient that does not impair the composition's therapeutic or prophylactic efficacy and/or can comprise a substance that augments or supplements the composition's efficacy.

In certain aspects, the compound of formula (I), or the pharmaceutically acceptable salt and/or solvate thereof, can be administered orally. In some aspects, the compound of formula (I), or the pharmaceutically acceptable salt and/or solvate thereof, can be administered in a capsule. In some aspects, the compound of formula (I), or the pharmaceutically acceptable salt and/or solvate thereof, can be administered in a tablet.

In certain aspects, the compound of formula (I), or the pharmaceutically acceptable salt and/or solvate thereof, can be formulated as described in U.S. Pat. No. 10,391,094, which is incorporated herein by reference. In some aspects, the compositions of the disclosure comprising the compound of formula (I) or the pharmaceutically acceptable salt and/or solvate thereof can comprise from about 50 mg to about 700 mg, about 75 mg to about 400 mg, or about 90 mg to about 200 mg of the compound of formula (I) or the pharmaceutically acceptable salt and/or solvate thereof. In some aspects, the compositions of the disclosure can comprise a compound of formula (I) or a pharmaceutically acceptable salt and/or solvate thereof in an amount of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 550 mg, about 600 mg, about 650 mg, or about 700 mg, or an amount ranging from and to any of these values. In some aspects, the compositions of the disclosure can comprise about 50 mg of a compound of formula (I) or a pharmaceutically acceptable salt and/or solvate thereof. In some aspects, the compositions of the disclosure can comprise about 100 mg of a compound of formula (I) or a pharmaceutically acceptable salt and/or solvate thereof. In some aspects, the compositions of the disclosure can comprise about 150 mg of a compound of formula (I) or a pharmaceutically acceptable salt and/or solvate thereof. In some aspects, the compositions of the disclosure can comprise about 50 mg of a compound of formula (Ia). In some aspects, the compositions of the disclosure can comprise about 100 mg of a compound of formula (Ia). In some aspects, the compositions of the disclosure can comprise about 150 mg of a compound of formula (Ia).

In some aspects, the composition of the compound of formula (I) can be formulated as shown in Table 1.

TABLE 1
Composition of Compound of Formula (I) 100 mg Hard Capsule

| | Composition | | | Reference |
|---|---|---|---|---|
| | Percentage | Per Unit | | to |
| Components | (%) | (mg) | Function | Standards |
| Compound of | 39.3 | 117.30 | Drug | In house |
| formula (I) | | (100.00) | substance | |
| Silicified | 59.7 | 178.45 | Diluent | NF |
| microcrystalline | | | | |
| cellulose high | | | | |
| density 90 μm^{a} | | | | |
| Sodium stearyl | 1.0 | 3.00 | Lubricant | NF, Ph, |
| fumarate | | | | Eur., JPE |
| Total capsule fill | 100.0 | 298.75 | — | — |
| mass | | | | |
| Brownish red | | 1 unit | Capsule shell | In house |
| opaque hard | | | | |
| gelatin capsule | | | | |
| (size #0) cap | | | | |
| with corporate | | | | |
| logo/body with | | | | |
| 5838 and 100 mg | | | | |
| printed in white | | | | |

^{a}Combination of Microcrystalline cellulose (NF, Ph. Eur., JP) & Silica, Colloidal Anhydrous (Ph. Eur.) eq. Colloidal Silicon Dioxide (NF) eq. Light Anhydrous Silicic Acid (JP)

In certain aspects, compositions of the present disclosure can be administered as the contents of a capsule containing the compound of formula (I). In some aspects, the contents of the capsule containing the compound of formula (I) can be dispersed in a nutritional supplement.

In some aspects, compositions of the present disclosure can be administered through a nasogastric tube, for example, when dispersed in a liquid such as a nutritional supplement

In certain aspects, compositions of the present disclosure can be administered from 1 to 10 times a day. In some aspects, the compositions of the present disclosure can be administered from 1 to 5 times a day. In some aspects, the compositions of the present disclosure can be administered 1, 2, 3, 4, 5, 6, 7, 8, 9, or 10 times a day. In some aspects, the compositions of the present disclosure can be administered once a day. In some aspects, the compositions of the present disclosure can be administered every other day. In some aspects, the compositions of the present disclosure can be administered once every other day. In some aspects, the compositions of the present disclosure can be administered every two days. In some aspects, the compositions of the present disclosure can be administered once every two days.

No clinically meaningful effect on the pharmacokinetics of fedratinib has been observed upon administration of fedratinib with a high-fat, high-calorie meal (approximately 815 calories of which 52% are derived from fat), and low-fat, low-calorie meal (approximately 162 calories of which 6% are derived from fat). Thus, fedratinib can be taken with or without regard to food. Moreover, the most frequently reported gastrointestinal (GI)-related treatment-emergent adverse events, such as nausea and vomiting, are less frequent in the fed state (62.5% and 16.7%,) than in the fasting state (87.5% and 66.7%) in subjects receiving a 500 mg fedratinib. Therefore, administration in the fed state, i.e., with nutritional supplement can reduce the incidence of nausea and vomiting.

In a population pharmacokinetics analysis of cumulative data from 452 patients, no clinically meaningful effect on fedratinib PK was observed with regard to age (20 years to 95 years), race, sex, body weight (40 kg to 135 kg), mild (defined as total bilirubin≤ upper limit of normal [ULN] and AST>ULN or total bilirubin 1 to 1.5 times ULN and any AST) or moderate (defined as total bilirubin>1.5 to 3 times ULN and any AST) hepatic impairment, and mild (defined as creatine clearance [CLcr]≥60 mL/min and <90 mL/min) renal impairment.

### EXAMPLES

### Example 1-Phase 1 Open-Label, Single-Center, 2-Part Crossover Study of Fedratinib Bioavailability in Healthy Adult Subjects

### Abbreviations

AE=adverse event; AUC=area under the plasma concentration-time curve; AUC (INF)=AUC from time zero extrapolated to infinite time; AUC (0-T)=AUC calculated from time zero to t, where t is the timepoint of the last quantifiable concentration; Cmax=maximum observed plasma concentration; ECG=electrocardiogram; ICF=informed consent form; IgG=immunoglobulin G; PK=pharmacokinetic; SAE=serious adverse event; BID=twice daily; NG=nasogastric; RBA=relative bioavailability.

### Study Objectives and Endpoints

TABLE 2
Study Objectives

Primary Objectives
The primary objectives of the study are:
Part 1:
To estimate the relative bioavailability (RBA) of 400 mg fedratinib in
healthy adult subjects when administered orally as contents of capsules
dispersed in a nutritional supplement in comparison to intact capsules
with a nutritional supplement.
Part 2:
To estimate the RBA of 400 mg fedratinib in healthy adult subjects when
administered as contents of capsules dispersed in a nutritional supplement
via nasogastric (NG) tube in comparison to intact capsules with a
nutritional supplement.
To estimate the RBA of 400 mg fedratinib in healthy adult subjects when
administered orally as intact capsules with a nutritional supplement as a
divided dose (200 mg twice daily [BID]) in comparison to a single dose of
intact capsules with a nutritional supplement.
Secondary Objectives
The secondary objectives of the study are:
Part 1:
To evaluate the safety and tolerability of a 400-mg dose of fedratinib in
healthy adult subjects when administered orally as contents of capsules
dispersed in a nutritional supplement or as intact capsules with a
nutritional supplement.
Part 2:
To evaluate the safety and tolerability of a 400-mg dose of fedratinib in
healthy adult subjects when administered orally as intact capsules with a
nutritional supplement; as contents of capsules dispersed in a nutritional
supplement via NG tube; or administered orally as intact capsules with a
nutritional supplement as a divided dose (200 mg BID).

TABLE 3
Study Endpoints

| Endpoint | Name | Description | Timeframe |
|---|---|---|---|
| Primary | Fedratinib PK: | Estimation of | All planned time |
| | Cmax | maximum observed | points throughout |
| | | plasma concentration | the study |
| | Fedratinib PK: | Estimation of AUC | All planned time |
| | AUC(0-T) | calculated from time | points throughout |
| | | zero to t, where t is the | the study |
| | | time point of the last | |
| | | quantifiable | |
| | | concentration | |
| | Fedratinib PK: | Estimation of AUC | All planned time |
| | AUC(INF) | calculated from time | points throughout |
| | | zero extrapolated to | the study |
| | | infinite time | |
| Secondary | Safety and | Adverse events, | Adverse events: |
| | tolerability | serious adverse events, | from the time ICF is |
| | | vital sign | signed until study |
| | | measurements, 12-lead | completion, and |
| | | ECG, clinical | when made known |
| | | laboratory safety tests | to the Investigator |
| | | (including hematology, | within 30 days after |
| | | chemistry, and urine | the last dose of |
| | | analysis), recording of | fedratinib |
| | | concomitant | Serious adverse |
| | | medications and | events: same |
| | | procedures | timeframe as for |
| | | | adverse events and |
| | | | at any time |
| | | | thereafter that are |
| | | | suspected of being |
| | | | related to fedratinib. |

### Study Design

An open-label, single-site, 2-part crossover study will be conducted in healthy adult subjects. This study will evaluate the RBA of 400 mg fedratinib when administered orally as contents of capsules dispersed in a nutritional supplement or via NG tube or administered orally as intact capsules with a nutritional supplement as a divided dose (200 mg BID) in comparison to intact capsules with a nutritional supplement. Each study part will consist of a randomized, multiple-sequence, open-label design. The study parts can be run in any order or in parallel. Subjects may participate in 1 part only. Each study part will consist of Screening, a Treatment phase (including baseline), and a Follow-up phone call. There will be a washout period of at least 12 days between each dose of fedratinib.

During the study, blood samples will be collected at prespecified times for PK. Subject safety will be monitored throughout the study. The study will be conducted in compliance with the International Council on Harmonization (ICH) of Technical Requirements for Registration of Pharmaceuticals for Human Use/Good Clinical Practice (GCP) and applicable regulatory requirements.

### Part 1:

Part 1 will consist of a randomized, 2-period, 2-sequence, open-label design. Subjects will be screened for eligibility. Subjects who meet all inclusion criteria and none of the exclusion criteria will return to the study center on Day-1 for protocol-specified assessments and the start of domiciling; subjects will remain domiciled at the study center throughout both study periods.

Subjects will be discharged on Day 23 upon completion of study procedures and satisfactory safety review. Each subject will receive a follow-up phone call approximately 4 days (+2 days) after discharge. In the event a subject discontinues from the study for any reason, an early termination (ET) visit will be performed. Only the safety assessments scheduled for the day of discharge should be performed at the ET visit. Each discontinued subject should also receive a follow-up phone call 4 days (+2 days) after completion of the ET visit.

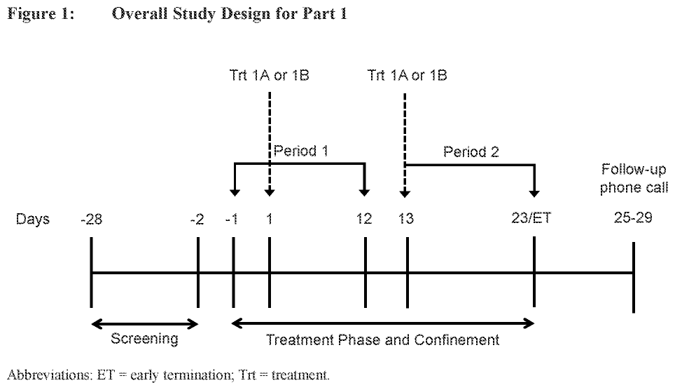

The overall study design is shown in FIG. 1.

### Part 2:

Part 2 will consist of a randomized, 3-period, 6-sequence, open-label design. Subjects will be screened for eligibility. Subjects who meet all inclusion criteria and none of the exclusion criteria will return to the study center on Day-1 for protocol-specified assessments and the start of domiciling; subjects will remain domiciled at the study center throughout all study periods.

Subjects will be discharged on Day 35 upon completion of study procedures and satisfactory safety review. Each subject will receive a follow-up phone call approximately 4 days (+2 days) after discharge. In the event a subject discontinues from the study for any reason, an ET visit will be performed. Only the safety assessments scheduled for the day of discharge should be performed at the ET visit. Each discontinued subject should also receive a follow-up phone call 4 days (+2 days) after completion of the ET visit.

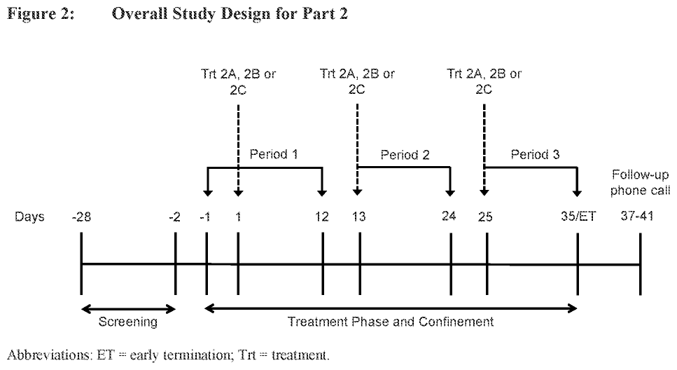

The overall study design is shown in FIG. 2.

### Study Duration for Subjects

### Part 1:

The estimated duration of each subject's participation, from screening through the follow-up phone call, is approximately 8 weeks.

### Part 2:

The estimated duration from screening through the follow-up phone call is approximately 10 weeks.

### End of Trial

The End of Trial is defined as either the date of the last visit of the last subject to complete the post-treatment follow-up, or the date of receipt of the last data point from the last subject that is required for primary, secondary and/or exploratory analysis, as respecified in the protocol, whichever is the later date.

### Treatment Administration and Schedule

On the evening before each dosing, all subjects will begin an overnight fast of at least 10 hours prior to dosing. No food or beverages (except water and any nutritional supplement administered with IP) will be allowed for at least 4 hours after dosing. Water is allowed as desired except for 1 hour before and 1 hour after each dosing. Each dose, with the exception of Treatment 2B, will be administered orally with approximately 240 mL noncarbonated, room temperature water. Dose modifications or interruptions are not permissible in this study.

Dose preparation and administration procedures for all treatment groups will be provided separately in a Study Manual or equivalent document.

### Part 1:

Each subject will receive the following treatments across 2 study periods:
Treatment 1A=oral administration of a single 400-mg dose of fedratinib (4×100-mg capsules) with a nutritional supplement (reference)
Treatment 1B=oral administration of a single 400-mg dose of fedratinib with contents of 4×100-mg capsules dispersed in a nutritional supplement (test)

All subjects will receive 8 mg oral ondansetron approximately 1 hour before each fedratinib administration to reduce the potential for fedratinib-related nausea and vomiting. A subsequent oral dose(s) of ondansetron may be given, as necessary, in accordance with the USPI instructions.

Subjects will be assigned randomly to 1 of 2 treatment sequences (N=14 per sequence); the sequence determines the order in which the treatments will be given (see Table 4).

TABLE 4
Treatment Sequences -Part 1

| | Number of | | |
|---|---|---|---|
| Sequences | Subjects (N) | Period 1 | Period 2 |
| 1 | 14 | 1A | 1B |
| 2 | 14 | 1B | 1A |

### Part 2:

Each subject will receive the following treatments across 3 study periods:
Treatment 2A=oral administration of a single 400-mg dose of fedratinib (4×100-mg capsules) with a nutritional supplement (reference)
Treatment 2B=a single 400-mg dose of fedratinib, administered via NG tube as contents from 4×100-mg capsules dispersed into a nutritional supplement (test)
Treatment 2C=oral administration of 400-mg fedratinib with a nutritional supplement, given as a divided dose (i.e., BID [2×100-mg capsules each]) (test)

Note: All subjects will receive 8 mg oral ondansetron approximately 1 hour before each fedratinib administration to reduce the potential for fedratinib-related nausea and vomiting. A subsequent oral dose(s) of ondansetron may be given, as necessary, in accordance with the USPI instructions.

Subjects will be assigned randomly to 1 of 6 treatment sequences (N=5 per sequence); the sequence determines the order in which the treatments will be given (see Table 5).

TABLE 5
Treatment Sequences - Part 2

| | Number of | | | |
|---|---|---|---|---|
| Sequences | Subjects (N) | Period 1 | Period 2 | Period 3 |
| 1 | 5 | 2A | 2B | 2C |
| 2 | 5 | 2A | 2C | 2B |
| 3 | 5 | 2B | 2A | 2C |
| 4 | 5 | 2B | 2C | 2A |
| 5 | 5 | 2C | 2A | 2B |
| 6 | 5 | 2C | 2B | 2A |

Prior to administration of Treatment 2B, a nasogastric tube will be placed and an X-ray(s) will be performed to confirm correct placement of the nasogastric tube. Following administration of Treatment 2B, the nasogastric tube will be flushed with a volume of sterile water for irrigation equal to approximately 60 mL. Lidocaine gel or a suitable therapeutic equivalent may be used to reduce participant discomfort during the nasogastric tube insertion procedure. The timing from start of the nasogastric tube placement to dosing will be up to 4 hours. Actual time of dose administration will be referred to as “0 hour.”

### Nutritional Supplement

All planned doses will be administered with a commercially available nutritional supplement (e.g., Ensure Plus® or Boost Plus®). The chosen nutritional supplement will be used throughout the study. The volume of nutritional supplement to be administered with each dose will be (e.g., approximately 180 mL).

### Required Concomitant Medications and Procedures

To reduce the potential for fedratinib-related nausea and vomiting, all subjects will receive 8 mg ondansetron orally approximately 1 hour before each fedratinib administration. A subsequent oral dose(s) of ondansetron may be given, as necessary, in accordance with the USPI instructions.

### Pharmacokinetic Analysis

Blood samples (approximately 3 mL each) for the measurement of fedratinib concentrations in plasma will collected at the following time points:
Treatment 1A, 1B, 2A and 2B: pre dose (0 hour) and at 0.5, 1, 1.5, 2, 3, 4, 6, 8, 12, 24, 48, 72, 120, 168, 192, and 240 hours post dose.
Treatment 2C: pre dose (0 hour) and at 0.5, 1, 1.5, 2, 3, 4, 6, 8, 12 (pre dose of the second dose), 12.5, 13, 13.5, 14, 15, 16, 18, 24, 48, 72, 120, 168, 192, and 240 hours post dose of the first dose.

Fedratinib plasma PK concentrations will be measured using a validated liquid chromatography tandem mass spectrometry assay

Fedratinib plasma PK parameters will be calculated using noncompartmental methods. The following key PK parameters will be estimated as allowed by the data:
Maximum observed plasma concentration (Cmax)
Area under the plasma concentration-time curve (AUC) from time zero to the last

quantifiable concentration (AUC [0-T])
AUC from time zero extrapolated to infinite time (AUC [INF])

The following plasma PK parameters, at minimum, will also be estimated as allowed by the data:
Time to Cmax (Tmax)
Apparent terminal plasma half-life (T-HALF)
Apparent total body clearance (CLT/F)
Apparent volume of distribution of terminal phase (Vz/F)

For Treatment 2C, Cmax and Tmax will be estimated both after the first dose and the second dose. AUC (0-T) and AUC (INF) will be estimated from time zero (the first dose) through 240 hours post-dose of the first dose.

Actual sampling times will be used for the calculation of all PK parameters, and additional PK parameters may be estimated, as deemed appropriate. Full details of the analysis methods will be documented in a statistical analysis plan.

### Statistical Analysis of Pharmacokinetic Data

The plasma concentrations and PK parameters will be summarized descriptively

(N, mean, standard deviation, coefficient of variation [CV %], geometric mean, geometric CV %, minimum, median, and maximum).

### Part 1:

To estimate the RBA for Treatments 1B in comparison with Treatment 1A, an ANOVA with treatment, period, and sequence as fixed effects, and subject nested within sequence as a random effect will be performed on the natural log-transformed Cmax, AUC (0-T), and AUC (INF). The geometric means along with ratios of the geometric means (test/reference) and associated 90% confidence intervals will be presented.

For Tmax, Wilcoxon signed-rank test, Hodges-Lehmann estimate, and its 90% CI will be calculated for the median difference between treatments.

### Part 2:

To estimate the RBA for Treatments 2B and 2C in comparison with Treatment 2A, an ANOVA with treatment, period, and sequence as fixed effects, and subject nested within sequence as a random effect will be performed on the natural log-transformed Cmax (Cmax after second dose for Treatment 2C), AUC (0-T), and AUC (INF). The RBA assessment will be based strictly on AUC only. The geometric means along with ratios of the geometric means (test/reference) and associated 90% confidence intervals will be presented.

For Tmax, Wilcoxon signed-rank test, Hodges-Lehmann estimate, and its 90% CI will be calculated for the median difference between treatments. For Treatment 2C, Tmax after second dose will be used for analysis.

### Pharmacogenetics

A pharmacogenetic (PG) blood sample (up to 10 mL) for potential analysis of deoxyribonucleic acid (DNA) related to drug metabolism and transport will be collected before dosing on Day 1.

### Example 2-Study Results

In Part 1, Cmax, AUC (0-T), and AUC (INF) were similar after a single dose of fedratinib administered orally as intact capsules (Treatment 1A, reference; n=27) or dispersed in a nutritional supplement (Treatment 1B, test; n=27). The ratio of geometric means (test/reference) showed no apparent difference in exposure for Cmax (108%), AUC (0-T) (102%), or AUC (INF) (103%).

In Part 2, compared with fedratinib capsules administered orally (Treatment 2A, reference; n=30), there was a slight reduction in exposures after a single dose of fedratinib administered via NG tube (Treatment 2B, test; n=30). The ratio of geometric means between test and reference showed<20% difference in exposures for Cmax (90%), AUC (0-T) (84%), or AUC (INF) (85%). Fedratinib administration in a divided dose (Treatment 2C, test; n=30) resulted in a reduction, as expected, in Cmax compared with a single dose of fedratinib (Treatment 2A, reference), with a 46% ratio of geometric means between test and reference. However, total exposure parameters AUC (0-T) measured as AUC (INF) showed<20% difference between test and reference (84% and 85% respectively).

No new safety signals or treatment-emergent serious adverse events were reported.

These data showed no significant differences in exposure to fedratinib 400 mg in healthy adult participants after a single dose administered with a nutritional supplement, via NG tube, or orally as a divided capsule dose, compared to a single dose of 4×100 mg capsules administered orally.

TABLE 6
Plasma Pharmacokinetics of Fedratinib by Administration Method

| | | Treatment 1B: | | Treatment 2B: | Treatment 2C: |
|---|---|---|---|---|---|
| | Treatment 1A: | fedratinib | Treatment 2A: | fedratinib | fedratinib |
| | fedratinib | 400 mg | fedratinib | 400 mg in | 2 × 200 mg |
| | 400 mg oral | dispersed in | 400 mg oral | supplement | capsules, |
| | capsules | supplement | capsules | via NG tube | divided dose |
| | n = 27 | n = 27 | n = 30 | n = 30 | n = 30 |
| Pharmacokinetic | geometric | geometric | geometric | geometric | geometric |
| Parameter | mean (CV %) | mean (CV %) | mean (CV %) | mean (CV %) | mean (CV %) |
| Cₘₐₓ, ng/mL | 1319 | 1425 | 1388 | 1246 | 641.4 |
| | (42.2) | (39.5) | (34.5) | (37.1) | (44.4) |
| AUC₀₋ₜ, h*ng/ml | 20127 | 20598 | 22683 | 19111 | 19002 |
| | (40.9) | (45.2) | (37.8) | (35.4) | (39.8) |
| AUC_{0-∞}, h*ng/ml | 22260 | 22838 | 24963 | 21334 | 21304 |
| | (40.1)^{a} | (42.1) | (38.9)^{a} | (34.0)^{a} | (40.3)^{a} |

^{a}One participant each in Treatment 1A, Treatment 2A, Treatment 2B, and Treatment 2C had pAUCe (percent extrapolated AUC) >20% and were excluded from AUC_{0-∞}. AUC₀₋ₜ, area under the plasma concentration-time curve from baseline to last quantifiable concentration; AUC_{0-∞}, area under the plasma concentration-time curve from baseline to infinity; Cₘₐₓ, maximum observed plasma concentration; CV, coefficient of variation.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. A method of treating a myeloproliferative disorder in a patient in need thereof, the method comprising administering to the patient an amount effective to treat a myeloproliferative disorder:
(a) a compound of formula (I): 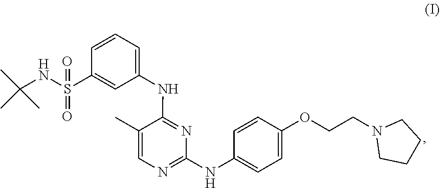 [SMILES: Cc1cnc(Nc2ccc(OCCN3CCCC3)cc2)nc1Nc1cccc(S(=O)(=O)NC(C)(C)C)c1]
or a pharmaceutical acceptable salt and/or solvate thereof; and
(b) a nutritional supplement.

2. The method of claim 1, wherein the solvate is a hydrate.

3. The method of claim 1 or 2, wherein the pharmaceutically acceptable salt is a hydrochloride salt or a hydrate thereof.

4. The method of any one of claims 1 to 3, wherein the dihydrochloride monohydrate of the compound of formula (I) is administered.

5. The method of any one of claims 1 to 4, wherein the compound of formula (I), or the pharmaceutically acceptable salt and/or solvate thereof, is formulated as a capsule.

6. The method of any one of claims 1 to 4, wherein the compound of formula (I), or the pharmaceutically acceptable salt and/or solvate thereof, is formulated by removing the contents of a capsule comprising the compound of formula (I) and dispersing it in a nutritional supplement.

7. The method of any one of claims 1 to 6, wherein the nutritional supplement is formulated as a powder, a suspension, a paste, a gel, a pudding, a solid, a liquid, or a liquid concentrate.

8. The method of any one of claims 1 to 7, wherein the compound of formula (I), or the pharmaceutically acceptable salt and/or solvate thereof, and the nutritional supplement are administered separately.

9. The method of claim 8, wherein the compound of formula (I), or the pharmaceutically acceptable salt and/or solvate thereof, and the nutritional supplement are administered sequentially within a time period of 15 minutes or less.

10. The method of any one of claims 1 to 7, wherein the compound of formula (I), or the pharmaceutically acceptable salt and/or solvate thereof, and the nutritional supplement are administered concomitantly.

11. The method of any of claims 1 to 7, wherein the compound of formula (I), or the pharmaceutically acceptable salt and/or solvate thereof, is dispersed in the nutritional supplement.

12. The method of claim 11, wherein the nutritional supplement is formulated as a liquid.

13. The method of any of claim 11 or 12, wherein the compound of formula (I), or the pharmaceutically acceptable salt and/or solvate thereof, is formulated as a powder.

14. The method of any one of claims 1 to 13, wherein the compound of formula (I), or the pharmaceutically acceptable salt and/or solvate thereof, and the nutritional supplement are administered orally.

15. The method of any one of claims 1 to 13, wherein the compound of formula (I), or the pharmaceutically acceptable salt and/or solvate thereof, is administered through a nasogastric tube.

16. The method of any one of claims 1 to 13, wherein the compound of formula (I), or the pharmaceutically acceptable salt and/or solvate thereof, and the nutritional supplement are administered through a nasogastric tube.

17. The method of any one of claims 1 to 16, wherein the method further comprises administering an anti-emetic compound.

18. The method of claim 17, wherein the anti-emetic compound is selected from droperidol, granisetron, metoclopramide, dexamethasone, bismuth subsalicylate, ondansetron, aprepitant, dolasetron, palonosetron, prochlorperazine, and rolapitant.

19. The method of claim 17 or 18, wherein the anti-emetic compound is ondansetron.

20. The method of any one of claims 17 to 19, wherein the anti-emetic compound is administered about 1 hour before the administration of the compound of formula (I), or the pharmaceutically acceptable salt and/or solvate thereof, and the nutritional supplement.

21. The method of any one of claims 17 to 20, wherein anti-emetic compound is administered at a dose of about 8 mg.

22. The method of any one of claims 17 to 21, wherein at least one additional dose of the anti-emetic compound is administered after the administration of the compound of formula (I), or the pharmaceutically acceptable salt and/or solvate thereof and the nutritional supplement.

23. The method of any one of claims 1 to 22, wherein the compound of formula (I), or the pharmaceutically acceptable salt and/or solvate thereof, is administered at a dose from about 50 mg to about 500 mg.

24. The method of any one of claims 1 to 23, wherein the compound of formula (I), or the pharmaceutically acceptable salt and/or solvate thereof, is administered at a dose of about 400 mg per day.

25. The method of any one of claims 1 to 23, wherein the compound of formula (I), or the pharmaceutically acceptable salt and/or solvate thereof, is administered once daily.

26. The method of any one of claims 1 to 25, wherein the compound of formula (I), or the pharmaceutically acceptable salt and/or solvate thereof, is administered twice daily.

27. The method of any one of claims 1 to 26, wherein the compound of formula (I), or the pharmaceutically acceptable salt and/or solvate thereof, is administered at a dose of about 200 mg twice daily.

28. The method of any one of claims 1 to 27, wherein the nutritional supplement does not comprise thiamine as the sole active ingredient.

29. The method of any one of claims 1 to 27, wherein the nutritional supplement comprises thiamine.

30. The method of any one of claims 1 to 29, wherein the nutritional supplement is a beverage.

31. The method of claim 30, wherein the beverage is a shake.

32. The method of any one of claims 1 to 31, wherein the patient is administered a compound of formula (Ia):

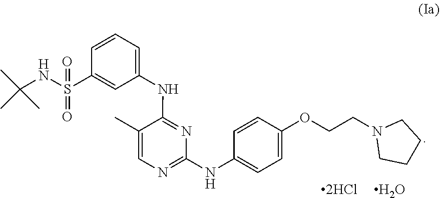

33. The method of any one of claims 1 to 32, wherein the myeloproliferative disorder is myelofibrosis.

34. The method of claim 33, wherein the myelofibrosis is primary myelofibrosis.

35. The method of claim 34, wherein the primary myelofibrosis is selected from intermediate risk primary myelofibrosis and high risk primary myelofibrosis.

36. The method of claim 33, wherein the myelofibrosis is secondary myelofibrosis.

37. The method of claim 33, wherein the myelofibrosis is post-essential thrombocythemia myelofibrosis.

38. The method of claim 33, wherein the myelofibrosis is post-polycythemia vera myelofibrosis.

39. The method of any one of claims 1 to 32, wherein the myeloproliferative disorder is acute myeloid leukemia (AML).

40. The method of any one of claims 1 to 32, wherein the myeloproliferative disorder is polycythemia vera.

41. The method of any one of claims 1 to 32, wherein the myeloproliferative disorder is essential thrombocythemia.
